(19) United States
(12) Patent Application Publication
(10) Pub. No.: US 2013/0178638 A1
(43) Pub. Date: Jul. 11, 2013

(54) PROCESS FOR PRODUCING DIOXOLANE

(71) Applicant: Sergio Mastroianni, Lyon (FR)

(72) Inventor: Sergio Mastroianni, Lyon (FR)

(73) Assignee: RHODIA OPERATIONS, Aubervilliers (FR)

(21) Appl. No.: 13/823,754

(22) PCT Filed: Sep. 7, 2011
(86) PCT No.: PCT/EP11/65438

§ 371 Date: Mar. 15, 2013

(30) Foreign Application Priority Data
Sep. 15, 2010 (FR) 1003666

(51) Int. Cl. C07D 317/20

(52) U.S. Cl. CPC C07D 317/20
USPC 549/453

(57) ABSTRACT

A process is described for producing dioxolane from crude glycerol obtained from raw materials such as the crude glycerol obtained during production of biodiesel or glycerol obtained during conversion of fats or oils. Further, the described method can dissolve the glycerol in an organic solvent and form an insoluble phase including salts included in the crude glycerol, and then react the glycerol obtained with an aldehyde or a ketone.

The present invention relates to a process for producing dioxolane from crude glycerol obtained from raw materials such as the crude glycerol obtained during the production of biodiesel or glycerol obtained during the conversion of fats or oils. The invention in particular aims to dissolve the glycerol in an organic solvent and to form an insoluble phase comprising the salts included in the crude glycerol, and then to react the glycerol obtained with an aldehyde or a ketone.

### PRIOR ART

It is a well-known practice to produce a dioxolane from glycerol and a ketone or an aldehyde. This reaction is in particular mentioned in the following publications: R. J. Fessenden & J. F. Fessenden, Organic Chemistry, Second Edition, Page 524, 1982 and T. W. Greene, Protective Groups in Organic Chemistry, John Wiley & Sons, 1981.

It is, however, more difficult to produce a dioxolane using crude glycerol. Glycerol, 1,2,3-propanetriol, is present in the combined form in vegetable and animal oils and fats. It is in particular present in the form of triglycerides combined with fatty acids such as stearic acid, oleic acid, palmitic acid and lauric acid. The most widely used industrial process for obtaining glycerol from vegetable and animal oils and fats involves saponification, high-pressure hydrolysis and transesterification reactions with alcohols, such as ethanol or methanol.

Glycerol is also a by-product of biodiesel which is generally obtained by transesterification of glycerides with short-chain alcohols, for example methanol or ethanol.

The transesterification reaction is catalyzed by an acid or a base, depending on the characteristics of the oils and/or fats used. After the transesterification reaction, the resulting esters are separated from the excess reagents, from the catalyst and from the by-products via a process comprising two steps. First, the glycerol is separated by settling out or centrifugation, and then the soaps and the residues of catalyst and of alcohol are removed by washing with water and sparging or the use of magnesium silicate with filtration. The large production of biodiesel as an alternative to fossil sources is accompanied by a high production of crude glycerol obtained as a by-product.

Depending on the production processes, the crude glycerol obtained comprises impurities, such as glycerides and salts, which involve numerous and complex treatment steps before being able to be used to produce dioxolane.

The development of a simple and industrial process for producing dioxolane from crude glycerol, which is inexpensive and under customary temperature and pressure conditions and which makes it possible to obtain dioxolane of high purity that would be appropriate for a certain number of applications, is thus sought.

### INVENTION

It has now been demonstrated that it is possible to produce dioxolane from crude glycerol by means of a process which is simple to implement and efficient and which, moreover, does not modify the glycerol or its color. This process consists in adding to the crude glycerol an amount of organic solvent which will dissolve the glycerol and thus form an insoluble phase consisting of a heterogeneous mixture of salts and of some organic compounds. This insoluble phase will then be separated from the liquid medium and it will be possible to use the purified glycerol for the production of dioxolane. This process allows excellent purification and separation of the glycerol regardless of the type of crude glycerol used. A purity of greater than 95% of glycerol containing very low amounts of residual salts is in particular obtained, and the solvent used can perfectly well be recycled. The dioxolane obtained has a purity absolutely equivalent to a dioxolane conventionally produced from pure glycerol.

The present invention thus relates to a process for producing dioxolane, comprising at least the following steps:
(a) bringing together crude glycerol and at least one organic solvent comprising from 1 to 10 carbon atoms and comprising at least one ketone, aldehyde, alcohol, acetal and/or ketal function, so as to form a liquid phase comprising the glycerol dissolved in the solvent(s) and to also form an insoluble phase;
(b) separating the insoluble phase and the liquid phase;
(c) optionally separating the solvent and the glycerol from the liquid phase;
(d) adding to the liquid phase or to the glycerol (if step (c) is carried out) a catalyst, and optionally a ketone or an aldehyde, so as to form a dioxolane by catalyzed reaction between the glycerol and the ketone or the aldehyde; and
(e) recovering the dioxolane.

The insoluble phase is generally a heterogeneous dispersed phase in the majority phase, and can be likened to a precipitate.

The process of the invention can be carried out continuously or batchwise. The steps mentioned may or may not be carried out successively and one after the other. Each of the steps of the process can be carried out continuously or batchwise.

The crude glycerol is preferentially obtained from renewable raw materials, in particular the crude glycerol is obtained during the production of biodiesel or obtained during the conversion of fats or oils, in particular animal or vegetable fats or oils. The crude glycerol is generally obtained by saponification, transesterification and/or hydrolysis reaction of animal or vegetable fats or oils.

The crude glycerol generally comprises from 5% to 95% by weight of glycerol and in particular from 40% to 90% by weight of glycerol. The crude glycerol also comprises inorganic salts, glycerides, water and other organic compounds.

The crude glycerol can optionally be treated for the process of the invention, in particular, for example, by adjusting the pH, filtration or distillation. It is thus possible to filter the crude glycerol in order to remove the insoluble organic matter and/or to distill it, generally at temperatures between 100 and 120° C. at atmospheric pressure in order to remove water and the volatile compounds. Some or all of the water contained in the crude glycerol can also be evaporated off before the glycerol is dissolved in the solvent.

Step a) of the process according to the invention aims to dissolve the glycerol in the organic solvent and to form an insoluble phase comprising the salts of the crude glycerol.

One or more solvents can be used. The solvent according to the invention can in particular be a ketone, an alcohol, an aldehyde, an acetal and/or a ketal. Acetals are obtained by nucleophilic addition of an alcohol to an aldehyde in an acidic medium, followed by removal of water. Ketals are obtained by the same type of reaction carried out on ketones.

The ketones preferentially used are acetone, cyclohexanone, methylcylohexanone, cyclopentanone, methylcyclopentanone and methyl isobutyl ketone (MIBK). The aldehydes preferentially used are formaldehyde, acetaldehyde and furfuraldehyde. The alcohols preferentially used are ethanol, methanol and isopropanol. The ketals and acetals are preferentially dioxolanes such as 2,2-dimethyl-1,3-dioxolane-4-methanol (solketal) for example.

A mixture of organic solvents, such as a mixture of alcohol and ketone, in particular a mixture of acetone and ethanol, is especially preferred.

In step a), no catalyst capable of catalyzing a reaction between the glycerol and the organic solvent(s) of the medium, in particular no esterification catalyst, will be used.

Step a) can last between 2 minutes and 1 hour. It can be carried out at a temperature between 10 and 100° C. and in particular between 20 and 50° C. The pH during this step can be between 6 and 12 and preferentially between 7 and 12.

The weight ratio between the crude glycerol and the solvent (crude glycerol/solvent) depends in particular on the solubility of the glycerol in said solvent, and is for example preferentially between 1/1 and 1/50.

Step b) aims to separate the precipitate obtained in step a) from the liquid phase comprising the solvent and the dissolved glycerol. Filtration, settling out or centrifugation can in particular be performed.

Step c) aims to optionally separate the solvent and the glycerol which is dissolved in the solvent. To do this, an evaporation or a distillation can in particular be carried out.

In step c), separation of the water contained in the crude glycerol can also be carried out.

The evaporation will in particular consist in causing the organic solvent(s) to pass into the gas state so as to be able to recover the glycerol in the liquid state.

One or more distillation columns can be used for carrying out the distillation. It is in particular possible to distill the various compounds on one and the same distillation column by varying the temperature and, optionally, the pressure; for example, to carry out the distillation of the organic solvent and then increase the temperature so as to distill the glycerol. Temperatures between 60 and 190° C. and pressures between 2 and 1000 mbar are customarily used.

This step c) can in particular be used for purifying the glycerol and ridding the medium of an organic solvent which might intervene in the catalyzed reaction for formation of the dioxolane in step d).

Step d) aims to form the dioxolane by reacting the glycerol with an aldehyde or a ketone in the presence of a catalyst. This reaction can be carried out in the liquid phase comprising the glycerol or else with the glycerol from which the organic solvent(s) has (have) been removed if a step c) was carried out. It should be noted that a ketone or aldehyde can optionally be added, in particular if a step c) has been carried out and depending on the solvent(s) used in step a).

The ketones preferentially used are acetone, cyclohexanone, methylcyclohexanone, cyclopentanone, methylcyclopentanone and methyl isobutyl ketone. The aldehydes preferentially used are formaldehyde, acetaldehyde and furfuraldehyde. It is in particular possible to use, according to the invention, one or more ketones and/or aldehydes for reacting the glycerol in the reaction medium.

Depending on the process used, it is possible to use various proportions of glycerol and of ketone or aldehyde in the reaction medium. For example, in batchwise mode, a molar ratio of 1 to 5 of ketone or aldehyde relative to glycerol can be used. In a continuous process, it is possible, for example, to use glycerol in a loop and to add low proportions of ketone or aldehyde, in particular from 5 to 20 mol %.

The dioxolane formed generally corresponds to general formula (I) below:

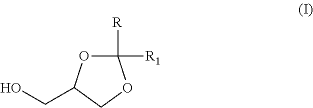

in which R and R₁ represent, independently of one another, a hydrogen atom or an alkyl chain comprising from 1 to 10 carbon atoms and in particular from 1 to 5 carbon atoms, such as, in particular, methyl, ethyl, propyl, isopropyl, butyl, isobutyl, pentyl and isopentyl groups.

In the case of a reaction with an aldehyde, one of the groups R or R₁ is a hydrogen atom. In the case of a reaction with a ketone, the groups R and R₁ do not represent a hydrogen atom.

The dioxolane formation reaction is generally carried out at a temperature between 0 and 150° C. and preferentially between 20 and 80° C.

This reaction can be carried out for 30 minutes to 8 hours, generally between 1 and 5 hours.

This reaction is preferentially carried out in an acidic medium, in particular with a pH ranging from 2.5 to 7.0.

Acid catalysts can in particular be used for this reaction, such as inorganic or organic acids or salts thereof. Mention may be made of the use of acetic acid, sulfuric acid, methanesulfonic acid and ion exchange resins of carboxylic or sulfonic type. These resins can be on a fixed bed in the reactor.

At the end of the reaction, it is preferable to neutralize the catalyst, in particular by adding a base. Mention may, for example, be made of sodium carbonate or sodium hydroxide.

The unreacted aldehyde and ketone can be removed by simple distillation.

It is possible to separate the dioxolane formed from the reaction medium by distillation, preferably under reduced pressure. One or more distillation columns can be used for carrying out the distillation. It is in particular possible to distill the various compounds on one and the same distillation column by varying the temperature and, optionally, the pressure; for example, to carry out the distillation of the ketone or of the aldehyde, then increase the temperature so as to distill the water, and then further increase the temperature so as to distill the dioxolane formed. Temperatures between 60 and 190° C. and pressures between 2 and 1000 mbar are customarily used.

A specific language is used in the description so as to facilitate understanding of the principle of the invention. Nevertheless, it should be understood that no limitation of the scope of the invention is envisioned by the use of this specific language. Modifications and improvements can in particular be envisioned by a person conversant with the technical field concerned on the basis of his own general knowledge.

The term “and/or” includes the meanings “and” and “or” and all the other possible combinations of elements connected with this term.

Other details or advantages of the invention will become more clearly apparent in the light of the examples given below purely by way of indication.

### EXPERIMENTAL SECTION

The commercially available crude glycerol has the following composition: 79.3%

by weight of glycerol, 15.8% by weight of water, 1.61% by weight of Na⁺ and 2.56% by weight of Cl⁻.

### Example 1

180.4 g of acetone are added to 15.13 g of crude glycerol at ambient temperature. The mixture is heated to 45° C. and stirred for 15 minutes until the glycerol has dissolved in the acetone and an insoluble phase has formed. The liquid phase is filtered using a PTFE filter with a pore diameter of 0.2 μm, and 191.23 g of clear solution are recovered. 307.9 mg of methanesulfonic acid are then added at ambient temperature and the mixture is stirred for 1 hour. The appearance of floculates is observed. 3.140 g of sodium carbonate are then added and the liquid phase is recovered by filtration. The volatile compounds are then evaporated off under vacuum at a temperature of 60° C. and a pressure of 0.3 bar absolute.

11.92 g of product are recovered, and said product is analyzed by various techniques:
2,2-dimethyl-1,3-dioxolane-4-methanol: 94.2% (gas chromatography)
water: 0.8% (gas chromatography)
glycerol: 5% (gas chromatography)
Na⁺: 0.07% (atomic absorption)
Cl⁻: 0.04% (argentometry)

The ketal can be further purified by distillation.

### Example 2

2464 g of acetone are added, at ambient temperature, to 120.2 g of crude glycerol. The mixture is stirred for 15 minutes until the glycerol has dissolved in the acetone and an insoluble phase has formed. The liquid phase is filtered using a PTFE filter with a pore diameter of 0.2 μm, and the solvent is evaporated off under vacuum at a temperature of 60° C. and a pressure of 0.3 bar absolute.

The amount of purified glycerol recovered is 88.93 g.

Analyses:
glycerol: 97.5% (GC)
water: 1.5% (GC)
Na⁺: 0.12% (atomic absorption)
Cl⁻: 0.18% (argentometry)
   Synthesis of 2,2-dimethyl-1,3-dioxolane-4-methanol

30.77 g of 97.5% glycerol obtained in the preceding step and 38.04 g of acetone at 40° C. (acetone/glycerol molar ratio=2) are introduced into a reactor with stirring. 95.7 mg of methanesulfonic acid are then added and the mixture is stirred for 1 hour. The gas chromatography analysis of the medium composed of a single perfectly homogeneous liquid phase shows a glycerol conversion of 56% and the corresponding formation of 2,2-dimethyl-1,3-dioxolane-4-methanol. The ketal can be separated from the mixture by distillation.

### Example 3

91.3 g of ethanol and 1754.3 g of acetone are added, at ambient temperature, to 150.8 g of crude glycerol. The mixture is stirred for 20 minutes until the glycerol has dissolved in the acetone and an insoluble phase has formed. The liquid phase is filtered using a PTFE filter with a pore diameter of 0.2 μm. The solvent is then evaporated off under vacuum at a temperature of 75° C. and a pressure of 0.3 bar absolute.

The amount of purified glycerol recovered is 106.7 g.

Analyses:
glycerol: 98.3% (GC)
Na⁺: 0.32% (atomic absorption)
Cl⁻: 0.46% (argentometry)
   Synthesis of 2,2-dimethyl-1,3-dioxolane-4-methanol

30.62 g of 98.3% glycerol obtained in the preceding step and 38.10 g of acetone at 40° C. (acetone/glycerol molar ratio=2) are introduced into a reactor with stirring. 94.6 mg of methanesulfonic acid are then added and the mixture is stirred for 1 hour. The gas chromatography analysis of the medium composed of a single perfectly homogeneous liquid phase shows a glycerol conversion of 54% and the corresponding formation of 2,2-dimethyl-1,3-dioxolane-4-methanol. The ketal can be separated from the mixture by distillation.

### Example 4

140.3 g of 2,2-dimethyl-1,3-dioxolane-4-methanol (commercial product) and 2190 g of acetone are added, at ambient temperature, to 140.7 g of crude glycerol. The mixture is stirred for 20 minutes until the glycerol has dissolved in the acetone and an insoluble phase has formed. The liquid phase is filtered using a PTFE filter with a pore diameter of 0.2 μm. The most volatile compounds are evaporated off under vacuum at a temperature of 75° C. and a pressure of 0.3 bar absolute.

The amount of glycerol/2,2-dimethyl-1,3-dioxolane-4-methanol mixture is 221.25 g.

Analysis:
glycerol: 44.2% (GC)
2,2-dimethyl-1,3-dioxolane-4-methanol: 55.5% (GC)
Na⁺: 0.2% (atomic absorption)
Cl⁻: 0.15% (argentometry)
   Synthesis of 2,2-dimethyl-1,3-dioxolane-4-methanol

44.8 g of the purified glycerol/2,2-dimethyl-1,3-dioxolane-4-methanol mixture obtained in the preceding step and 25.3 g of acetone at 40° C. (acetone/glycerol molar ratio=2) are introduced into a reactor with stirring. 63.7 mg of methanesulfonic acid are then added and the mixture is stirred for 1 hour. The gas chromatography analysis of the medium composed of a single perfectly homogeneous liquid phase shows a glycerol conversion of 41% and the corresponding formation of 2,2-dimethyl-1,3-dioxolane-4-methanol. The ketal can be separated from the mixture by distillation.

### Example 5

### Comparative

Synthesis of 2,2-dimethyl-1,3-dioxolane-4-methanol using crude glycerol

37.86 g of crude glycerol (corresponding to 30.0 g of pure glycerol) and 38.04 g of acetone at 40° C. (acetone/glycerol molar ratio=2) are introduced into a reactor with stirring. 95.4 mg of methanesulfonic acid are then added and the mixture is stirred for 1 hour. The gas chromatography analysis of the medium, corresponding to a mixture of two liquid phases and of a white precipitate (predominantly composed of NaCl), shows a glycerol conversion of only 17% and the corresponding formation of 2,2-dimethyl-1,3-dioxolane-4-methanol.

1. A process for producing dioxolane, the process comprising the following steps:
(a) bringing together crude glycerol and at least one organic solvent comprising from 1 carbon atom to 10 carbon atoms and comprising at least one of a ketone, an aldehyde, an alcohol, an acetal and/or a ketal function, so as to form a liquid phase comprising the-glycerol dissolved in the solvent(s) and to also form an insoluble phase;
(b) separating the insoluble phase and the liquid phase;
(c) optionally separating the solvent and the glycerol from the liquid phase;
(d) adding to the liquid phase or to the glycerol (if step (c) is carried out) a catalyst, and optionally a ketone or an aldehyde, so as to form a dioxolane by catalyzed reaction between the glycerol and the ketone or the aldehyde; and
(e) recovering the dioxolane.

2. The process as defined by claim 1, wherein the crude glycerol originates from a renewable raw material.

3. The process as defined by claim 1, wherein the crude glycerol is obtained during production of biodiesel or obtained during conversion of a fat or an oil.

4. The process as defined by claim 1, wherein the ketone is selected from the group consisting of an acetone, a cyclohexanone, a methylcylohexanone, a cyclopentanone, a methylcyclopentanone and a methyl isobutyl ketone.

5. The method as defined by claim 1, wherein the aldehyde is selected from the group consisting of a formaldehyde, an acetaldehyde and a furfuraldehyde.

6. The method as defined by claim 1, wherein the alcohol is selected from the group consisting of an ethanol, a methanol and an isopropanol.

7. The method as defined by claim 1, wherein the ketal or the acetal is a dioxolane.

8. The method as defined by claim 1, wherein the weight ratio between the crude glycerol and the solvent (crude glycerol/solvent) is from 1/1 to 1/50.

9. The method as defined by claim 1, wherein the separation in step b) is carried out by filtration.

10. The method as defined by claim 1, wherein the separation in step c) is carried out by evaporation.

11. The method as defined by claim 1, wherein the dioxolane corresponds to general formula (I) below: 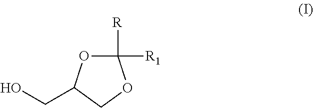
in which R and R₁ represent, independently of one another, a hydrogen atom or an alkyl chain comprising from 1 carbon atom to 10 carbon atoms.

12. The process as defined by claim 1, wherein the reaction medium of step d) comprises an acid catalyst.

13. The process as defined by claim 1, wherein the reaction medium of step d) comprises an acid catalyst selected from the group consisting of acetic acid, sulfuric acid, methanesulfonic acid, a carboxylic ion exchange resin and a sulfonic ion exchange resin.

14. The process as defined by claim 3, wherein the fat or oil is an animal or vegetable fat or oil.
